(19) United States
(12) Patent Application Publication
(10) Pub. No.: US 2026/0216456 A1
(43) Pub. Date: Jul. 30, 2026

(54) Negative Pressure Airway Clearance System with Adaptive Suction Control and Patient-Specific Safety Mechanisms

(71) Applicants: Emerald Wang, Carmel, IN (US); Chi Wang, Carmel, IN (US); Katherine Wang, Carmel, IN (US)

(72) Inventors: Emerald Wang, Carmel, IN (US); Chi Wang, Carmel, IN (US); Katherine Wang, Carmel, IN (US)

(21) Appl. No.: 19/574,492

(22) Filed: Mar. 23, 2026

Related U.S. Application Data
(63) Continuation-in-part of application No. 18/911,945, filed on Oct. 10, 2024, now Pat. No. 12,599,713.

(51) Int. Cl. A61M 16/00; A61M 16/06

(52) U.S. Cl. CPC A61M 16/0009; A61M 16/024; A61M 16/0605; A61M 2202/0014

(57) ABSTRACT

A portable airway clearance system utilizing adaptive suction control, modular architecture, and intelligent patient profiling is disclosed. The system includes a suction mechanism configured to generate negative pressure, an airflow pathway, and control circuitry configured to modulate suction according to a time-varying suction profile. The system supports multiple suction generation mechanisms, including diaphragm pumps and propeller systems, and utilizes nonlinear waveform modulation for effective and safe airway obstruction removal. In certain embodiments, sensor inputs are used to dynamically adjust or terminate suction, and patient classification is used to constrain operation within predefined safety limits.

### CROSS REFERENCE TO RELATED APPLICATIONS

This application is a Continuation-in-Part of U.S. patent application Ser. No. 18/911,945, filed Oct. 10, 2025, the entire contents of which are incorporated herein by reference.

### FIELD OF THE INVENTION

The present invention relates to medical devices for airway obstruction removal, and more particularly to portable negative pressure airway clearance systems with adaptive suction control, modular airflow configurations, and intelligent patient-specific safety mechanisms.

### BACKGROUND OF THE INVENTION

Choking is a critical and life-threatening emergency that requires immediate intervention. It can occur in various environments such as homes, restaurants, and public spaces, often affecting individuals of all ages. According to medical statistics, airway obstructions are a leading cause of unintentional injury-related deaths, especially in young children and the elderly. For example, US National Safety Council reported that in 2022, over 5500 deaths due to choking, ranked the fourth leading causes of unintentional injury related deaths [1]. Traditional methods, such as the Heimlich maneuver, are commonly used to expel blockages from the airway. However, these methods have limitations, particularly in situations where the individual is alone, incapacitated, or where physical strength or technique is inadequate to generate the required force to clear the airway.

In recent years, mechanical suction devices have been introduced as alternatives to manual methods. These devices typically use negative pressure to dislodge the blockage by pulling it away from the airway. However, many existing devices present several drawbacks [2]:
Difficult to operate and unreliable suction: Most of the current devices are manual, requiring both hands applying forces in different directions and generating unreliable suction pressure, sometimes too weak or too strong, depending on the rescuer's skills and strength.
Lack of adaptability: Current devices, manual or automatic, offer only a single, continuous suction pattern/strength, which may not be effective or even increase the risk of injury based on patient characteristics such as age, size, or the nature of the obstruction.
Safety concerns: Devices that generate uncontrolled or excessive negative pressure can cause damage to the airway, especially in vulnerable patients like babies and the elderly. Without adequate safety mechanisms, these devices can collapse the airway or cause soft tissue damage. [2] [3]

The present invention addresses these challenges by introducing a modular, portable anti-choking device with embedded control programs that:
Provides multiple suction patterns (e.g., pulse and intermittent suction) to better mimic the natural forces involved in coughing as trying to dislodge obstructions.
Incorporates real-time sensor feedback to dynamically adjust suction strength, ensuring both effectiveness and safety across a wide range of patient types.
Includes safety mechanisms to prevent over-suction and minimize the risk of tissue damage.

The enhanced design offers an effective, reliable, and safe solution for airway obstruction emergencies for all patient types.

### DESCRIPTION OF PRIOR ART

Existing anti-choking devices, whether manual or automatic, have many limitations. With portable manual devices, weak and/or inconsistent suction and cumbersome use are among top reported shortcomings from consumer feedback. Recently commercialized manual devices require the use of both hands, applying forces in different directions (one on mask and one pulling the plunger), and some with small vacuum air chamber creates limited suction, while others with strong suction pose a potential risk of damaging airway tissues. Current portable automatic devices utilize an electric fan or a spring. Insufficient suction power is the most sited drawback by consumers. Additionally, larger electric devices like U.S. Pat. No. 5,609,149 and US 20230372600 offer limited portability due to their bulky size, making them less practical for use outside clinical settings.
US20240066280 (Feb. 29, 2024): This invention utilizes manually created vacuum suction but requires both hands pushing (the mask down) and pulling (the plunger) in opposite directions, making it difficult for a single individual to operate effectively and often requires additional personnel to assist the patient. The operation also yields variable suction depending on the rescuer's skills and strength, limiting its effectiveness.
U.S. Pat. No. 11,478,575 (Oct. 25, 2022), U.S. Pat. No. 11,701,462 B2 (Jul. 18, 2023), U.S. Pat. No. 11,759,591 (Sep. 19, 2023) work in similar manners: they utilize manually created vacuum suction with similar limitations of unreliable suction pressure from too weak to too strong.
US20230053877 (Feb. 23, 2023): This invention utilizes suction effect created by a mechanical spring. It can be operated by one hand but has one-level suction power.
US20230372600 (Nov. 23, 2023): This invention utilizes suction effect created by airflow dynamics. It is complicated and bulky.
WS 2021/1266698: is a fluid drainage line with a pump and a venting one-way valve to prevent airlocks. Its claims are about drainage lines/catheters and venting.
U.S. Pat. No. 5,609,149 (Mar. 19, 1997): This invention uses a manually operated plunger to provide the suction effect, which is bulky and offers inconsistent effectiveness.
U.S. Pat. No. 8,876,838 (Nov. 4, 2014): This invention uses compressed fluid for suction but requires additional effort for repeated use.

### SUMMARY OF THE INVENTION

The present invention relates to a modular, portable airway clearance device configured to generate controlled negative pressure for removing airway obstructions. The invention addresses limitations of existing anti-choking devices by providing enhanced suction control, adaptable safety mechanisms, and flexible system architectures suitable for a wide range of patient types and emergency environments.

In certain embodiments, the device includes a suction mechanism configured to generate negative pressure, wherein the suction mechanism may comprise a propeller-driven system, a diaphragm pump, a piston pump, a Venturi-based vacuum generator, or combinations thereof. The suction mechanism is controlled by embedded computing programs to dynamically regulate suction strength and patterns based on user input and real-time sensor feedback.

The device provides multiple suction patterns, including pulse suction, intermittent suction, hybrid suction, and generalized waveform-based suction profiles. In certain embodiments, suction is modulated using nonlinear periodic functions, including asymmetric waveforms with variable duty cycles, frequencies, and amplitudes. These suction profiles are configured to mimic or enhance natural coughing dynamics, thereby improving the effectiveness of obstruction dislodgement while reducing the risk of airway injury.

In certain embodiments, the device incorporates intelligent control programs configured to dynamically adjust suction strength based on real-time sensor feedback. The system may monitor parameters including air pressure, airflow, and object motion within the airflow pathway. The control programs may automatically adjust suction intensity, waveform characteristics, or terminate operation upon detection of airway clearance or unsafe conditions.

In certain embodiments, the device includes an intelligent patient profiling system configured to determine patient type based on measured airway response. The system may apply an initial micro-pulse suction and determine airway resistance, compliance, or airflow characteristics. Based on these measurements, the device may automatically classify the patient into categories such as neonate, infant, child, or adult, and constrain suction within predefined clinical safety ranges.

The device may incorporate multiple sensing modalities, including pressure sensors, motion sensors, acoustic sensors, and optical sensors. In certain embodiments, sensor fusion techniques are used to improve detection of obstruction movement or clearance events, enabling more accurate and responsive control of suction operation.

In certain embodiments, the airflow pathway is modular and configurable. The airflow path may be linear, curved, or otherwise arranged, and may optionally exclude debris filters or include alternative debris capture mechanisms such as removable cartridges or external collection chambers. In some embodiments, airflow may travel substantially straight from the facemask to an outlet without requiring significant directional changes.

The device includes a modular architecture allowing components such as the facemask, airflow channels, suction module, and control module to be assembled, disassembled, or reconfigured. This modular design improves portability, maintenance, and adaptability across different use cases.

In certain embodiments, the device includes user interface elements allowing selection of patient type, suction strength, and suction pattern. In other embodiments, these parameters may be automatically determined by the control system without user intervention.

Safety mechanisms are integrated to prevent excessive suction and minimize risk of tissue damage. These mechanisms may include pressure thresholds, automatic shutoff conditions, and adaptive control algorithms that maintain suction within clinically acceptable ranges for different patient populations.

In certain embodiments, the device further comprises one or more filter elements configured to capture or intercept debris, fluids, or particulate matter dislodged from the airway during suction. The filter elements may be positioned along the airflow pathway between the facemask and the suction mechanism and may include primary and secondary filters configured to prevent debris from entering internal components of the device. The filters may be formed from flexible or rigid materials and may be removable, replaceable, washable, or disposable. In certain embodiments, the filters are configured to maintain airflow while trapping debris, thereby preserving suction efficiency and protecting internal components. In alternative embodiments, the device may operate without filters or may utilize alternative debris management mechanisms, including external collection chambers or inline capture modules.

The device may further include a dual power system configured to operate using AC power or rechargeable batteries, enabling use in a variety of environments including homes, restaurants, public spaces, and remote locations.

In summary, the present invention provides a versatile, intelligent, and safe airway clearance system that combines adaptive suction control, generalized waveform modulation, multi-modal sensing, and modular design. The invention enables improved effectiveness in removing airway obstructions while maintaining safety across diverse patient populations and operating conditions.

### BRIEF DESCRIPTION OF THE DRAWINGS

FIG. 1 is a perspective view of key modules and components of an embodiment of the invention with a propeller and with airflow passing through a handle.

FIG. 2 is a perspective view of the assembled view and an action view of an embodiment of the invention with airflow passing through a handle.

FIG. 3 contains a side section view and a bottom view of an embodiment of the invention with a propeller and with airflow passing through a handle.

FIG. 4 contains the front view and rear view of an embodiment of the invention with a handle.

FIG. 5 contains suction workflows of air pressure sensor and motion sensor of an embodiment of the invention.

FIG. 6 illustrates an embodiment of the invention with a linear airflow pathway from the facemask straight to the distal end of the device and with a diaphragm pump as a suction mechanism.

FIG. 7 is a perspective view of key modules and components of an embodiment of the invention with a diaphragm pump and with airflow passing through a handle.

FIG. 8 contains the AI-driven Patient Profiling workflows.

FIG. 9 contains the Sensor Fusion Decision Logic workflows.

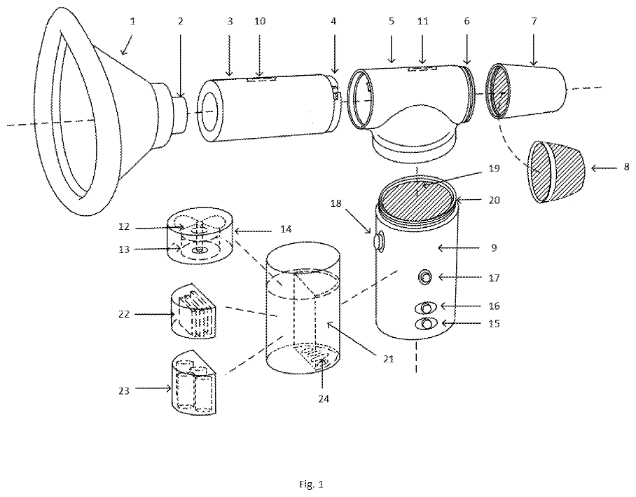

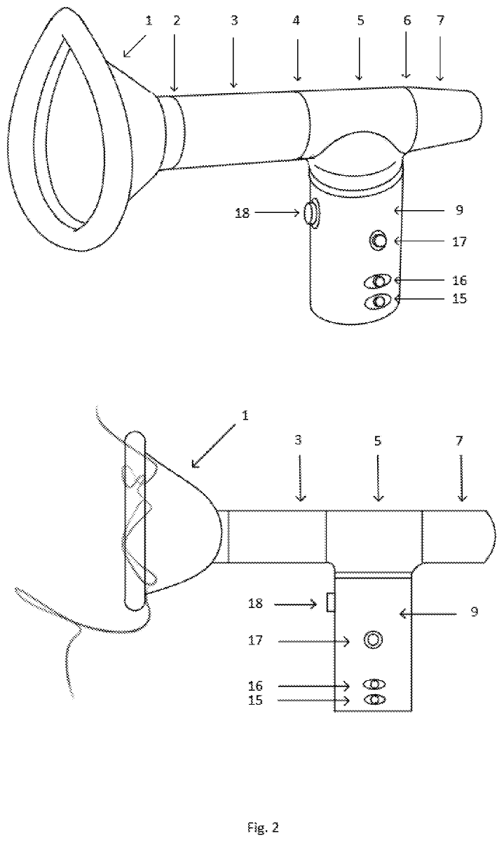

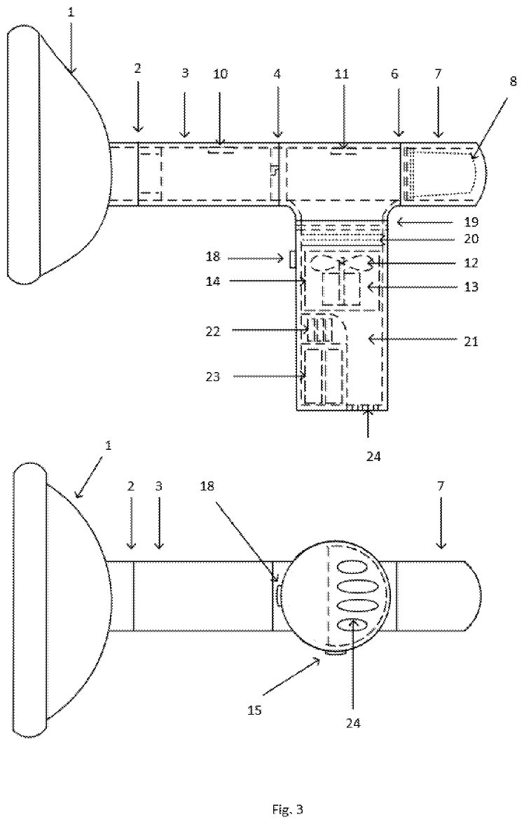

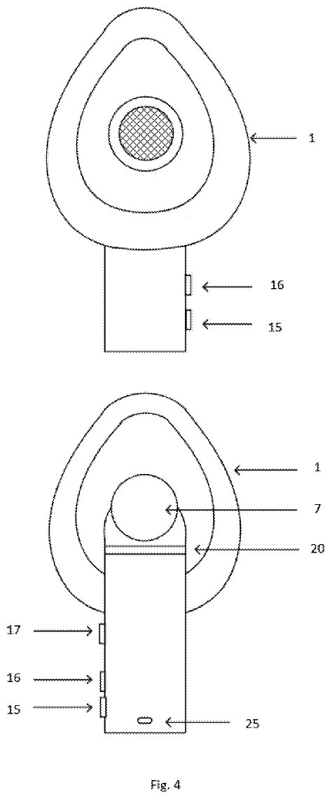

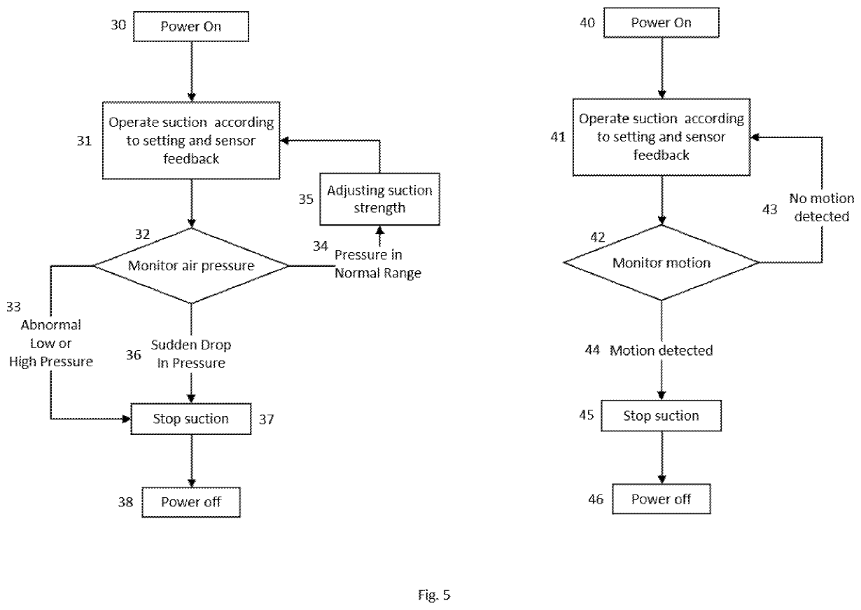

### DETAILED DESCRIPTION OF THE INVENTION

The anti-choking device comprises multiple innovative features that enhance its functionality, portability, and safety. These include:
Suction Mechanism: The device uses a high-velocity propeller driven by a motor to generate sufficient suction power for airway clearance. The propeller's speed and suction pattern are controlled by embedded algorithms tailored to the patient type and needs.
Multiple Suction Modes: Multiple suction modes are provided:
   1. Pulse Suction: This mode applies varying suction strength over time, controlled by a sinusoidal or triangular wave function. It is particularly effective for dislodging obstructions by creating a rocking or shifting motion of the object.
   2. Intermittent Suction: This mode alternates between continuous suction and brief pauses. The cycle is adjustable based on patient type, with ramp-up and ramp-down times for smooth transitions.
   3. Hybrid Suction: This mode combines both pulsed and intermittent suction functionalities. The suction is applied in short, controlled bursts (pulses), with pauses between each set of pulses to create intermittent cycles of activity and rest.
Control Programs for Enhanced Suction Power and Versatility: The device features advanced control program that dynamically adjusts suction patterns and strength in real time based on sensor feedback. These suction patterns mimicking the natural coughing reflex can deliver more tailored suction power. This flexibility allows the device to handle a wide range of obstruction types with stronger and more stable suction, ensuring a higher success rate in dislodging obstructions safely and efficiently.
Sensor Feedback: Integrated pressure and motion sensors provide real-time monitoring of the device's operation. The feedback from the sensors allows the device to adjust suction operation dynamically for stronger and versatile suction. It ensures that the suction remains within safe limits and stops the device immediately once an obstruction is cleared or if abnormal conditions (e.g., excessive pressure or leaks) are detected.
Dual Power Supply: The device features a dual power option, enabling it to operate either with an AC power source or a rechargeable battery pack. This flexibility allows for uninterrupted operation in any environment.
User Customization: The device provides control options for selecting suction patterns suction strength levels, and patient types, ensuring that the suction is appropriate for different age groups and patient anatomies. The user interface allows easy switching between suction modes, offering adaptability to various emergency situations.

### 1. Facemask and Air Chambers

In the event of a choking emergency, the facemask (1) is placed securely over the patient's face. The facemask (1) will fit snugly cover the patient's mouth and nose, forming an airtight seal necessary for effective suction. The facemask (1) connects to the air chamber (3) via a push-fit locking mechanism (2), enabling quick attachment and detachment. The air chamber (3) connects to air chamber (5) via a locking mechanism (4). The air flow continues through air chamber (5) then drawn into air chamber (21) inside of the power module (9) by the propeller-motor unit (14), and exits from outlets (24). The propeller-motor unit (14) contains a high-velocity propeller (12) driven by a motor (13) to allow the propeller (12) to create negative suction. Quick-release mechanisms (4), (6), and (20) enable easy assembly, disassembly for cleaning. The facemask utilized in this invention can be sourced from commercially available providers. All air chambers (3, 5, 21) and the body of the power module (9) are made from durable materials such as suitable plastic or metal, easy for cleaning and reassembly.

### 2. Filters

The device uses one or more filters to prevent debris and large particles to enter the power module (9). In an embodiment, a deep pocket shaped filter (8) is placed in the filter cabinet (7). The secondary filter (19) is located at the entrance of the power module (9). Both filters (8) and (19) can be easily mounted and unmounted, allowing for quick cleaning between uses, after the device is disassembled at locking mechanism (6) and (20). Filter (8) is made from flexible materials such as nylon, foam, or other suitable materials that are absorbent, washable/reusable or disposable, designed to absorb/trap small debris and liquid. Filter (19) is made from durable plastic or metal to stop large particles entering the power module (9).

### 3. Monitoring Sensors

The device uses pressure and motion sensors to monitor suction status and provide feedback to the control system, enabling intelligent management of the suction operation. In an embodiment, sensors (10) and (11) are positioned before and around filters (8) and (19) within the air chambers (3) and (5), continuously tracking air pressure and the movement of extracted objects from the patient's throat. Depending on the embodiment, various sensor types may be employed for monitoring suction status and detecting extracted objects. Motion sensors detect the objects, while pressure sensors identify changes in pressure caused by the dislodging of obstructions or issues such as facemask leakage or removal. These sensors, used individually or in combination, provide real-time feedback to the control program, allowing for automatic adjustments to suction strength or immediate cessation when the obstruction is cleared or the suction force becomes excessive, ensuring safety and reduce the risk of airway injury.

The sensors utilized in this device, including pressure and motion sensors, are selected from readily available, commercial-grade components to ensure both performance and affordability. Careful calibration of the sensors ensures precise measurements of suction force and air pressure, while also preventing overuse or excessive force that could pose risks to the patient's airway.

### 4. High-Velocity Propeller Module

The propeller-motor module (14), located in the power module (9), is the core component of the suction mechanism. The air propeller (12) is selected from currently available models. The motor (13), which drives the propeller, is a commercially available high-efficiency DC motor, selected for its reliability, compact size, and ability to handle rapid speed changes. The motor is powered by either an AC power source through the charging port (25) or by batteries located in the battery module (23), with power switching managed by an intelligent power management integrated circuit (PMIC) in the control module (22). The motor speed, which dictates both suction strength and suction patterns, is controlled by embedded software in the control circuit. This software dynamically adjusts the motor based on selected patient type (such as baby, child, adult, or elderly), suction range for selected patient type, suction pattern, and real-time feedback from integrated sensors (10), (11). The combination of versatile settings and intelligent control ensures that the propeller-motor module operates effectively and safely, adapting to various patient needs, including children and adults, while maintaining consistent performance.

### 5. Dual Power Supply

In an embodiment, the power management system including an intelligent power management integrated circuit (PMIC) in the control module (22) with integrated software to manage the power supply:
Automatic Power Source Switching: The power management system will automatically switch between AC power and battery power as needed, ensuring continuous operation.
AC Power Compatibility: When connecting the charging port (25) of the device to an AC outlet, the device operates continuously without drawing power from the battery.
Power Efficiency and Safety: In an embodiment, the power supply design incorporates features such as overcharge protection, short-circuit protection, and automatic shutdown to prevent damage to the device and ensure patient safety.
Rechargeable Battery Pack: The rechargeable battery pack (23) provides a reliable power source during travel or in settings without easy access to electrical outlets.

### 6. Control Circuits and Embedded Control Programs

The device includes the control module (22) utilizing advanced control circuits that host essential computing programs to manage its operation. The circuits incorporate a Power Management Integrated Circuit (PMIC), which efficiently handles switching between power sources and optimizes battery usage. These circuits also include a motor driver, acting as the interface between the motor and the device's control software, ensuring smooth operation. The control software, embedded in the circuits, is designed to dynamically manage the suction operation by communicating with sensors and the motor driver. It continuously monitors sensor feedback, such as air pressure and motion within the airway, and adjusts motor speed to modify suction strength in real time. The software is powered by intelligent algorithms, enabling it to automatically alter the suction pattern and strength or stop suction altogether, based on real-time data from the sensors. This adaptability ensures safe and effective operation across different patient types and emergency scenarios.

These control circuits and embedded programs are housed within the control module (22), which is constructed from durable materials like heat-resistant plastics or aluminum to protect the sensitive electronics from external damage or environmental conditions. The circuits are commercially available, off-the-shelf components that meet industry standards for medical devices, ensuring ease of sourcing and replacement if necessary.

### 7. User Interface Control Options for Flexibility and Safety

On and Off Switch: When the facemask (1) is correctly positioned and sealed, the user activates the suction function using a power button or a switch. In an embodiment, the user can turn-on or turn-off the device on using the power control button (18).

Patient Type Selector: The device has interface element to let users set patient type, such as babies, children, and adults. Since the safe range of suction strength for different patients varies, this setting makes the device safer and better suits various scenarios. For example, for oropharyngeal or nasopharyngeal suction, medical references suggest safe rage of portable unit for neonates to be between 60-80 mmHg, infants 80-100 mmHg, children 100-120 mmHg, adults 100-150 mmHg. [4] The younger the patient, the risk of damaging soft tissues or airway collapse of damage to mucous membranes may be higher. This patient type option allows the device to be adapted for a wide range of application scenarios and ensuring safety of different types of patients. The control program will automatically adjust maximum suction strength according to this setting. In an embodiment, user can use switch (15) to select patient type. In other embodiments, the device may have other forms of control interfaces for this option.

Suction Strength Selector: The device has interface element to let users select different levels of suction strength. For each type of patient, the device will perform suction with strength within its safe range. For safety, the device may start with suction strength at the lower end of established sage range and gradually increase when needed. For patients of larger size in the same patient type, to save time, user may select to start with a higher strength within the safety range. In an embodiment, user can use interface element (16) to select suction strength. In other embodiments, the device may have other forms of control interfaces for this option.

Suction Pattern Selector: The device has interface element to let users select different suction patterns, including, not limited to, pulse suction, intermittent suction, and hybrid suction. In an embodiment, user uses interface element (17) to select suction pattern. In other embodiments, the device may have other forms of control interfaces for this option:
Pulse Suction: Pulse suction creates suction in bursts with pressure changes in periodic patterns. Pulsed suction is potentially more effective and safer for extracting choking obstructions because it mimics a natural coughing reflexes “pumping” action, which might allow the obstruction to be dislodged more effectively, as opposed to continuous suction. The benefits of pulsed suction include:
   Dynamic Pressure Changes: Pulsing creates alternating pressure gradients. This variation could apply intermittent force to the obstruction, loosening it by “rocking” or shifting it slightly with each pulse. This rocking motion may free the obstruction more effectively than a steady pull.
   Reduced Risk of Collapse: Continuous suction might create a high vacuum that could cause soft tissues (like the airway walls) to collapse inward or damaging surrounding tissues after the obstruction is removed.
   Enhanced Clearance in Fluids: If mucus or liquid is involved along with the obstruction, pulsed suction may help clear these fluids more efficiently, as they can sometimes impede continuous suction by forming a seal around the device's tip.
   Higher Maximum Pressure: Pulsed suction may allow for higher maximum pressures without causing damage because it doesn't subject the tissues to constant stress.
Intermittent Suction: Intermittent suction is a cyclical suction pattern, where the device alternates between suction and brief pauses, but without the complex pressure variations in a pulse suction pattern. The benefits of intermittent suction include:
   Safety: The pauses between suction allow airway tissues to relax, preventing overexposure to continuous negative pressure, which could lead to tissue damage, airway collapse, or discomfort.
   Effectiveness: The brief pauses during intermittent suction can help the body react and reposition the obstruction, allowing a better “grip” on the blockage when suction resumes due to the slight release in pressure.
   Versatility: The on-off intervals can be adjusted to different patient types of obstructions, offering a customizable solution without the need for complex pressure control.
   Simplicity: This design is easier to implement compared to more complex pulse suction systems because it doesn't require variable suction strength, just timed intervals.
Hybrid Suction: In this mode, the suction is applied in short, controlled bursts (pulses), with pauses between each set of pulses to create intermittent cycles of activity and rest. This dual-mode operation is beneficial for applications requiring precision and reduced strain on materials that are sensitive to constant suction or excessive force. The pulse intensity, frequency, and the duration of intermittent pauses can all be adjusted to suit the specific requirements of the task, allowing for a highly customizable suction process. This combined mode ensures optimized efficiency by balancing the need for powerful suction bursts with intervals of reduced or no suction, thus preventing material damage or wear over time.

### 8. Models for Suction Pattern and Strength

In certain embodiments, suction applied by the device is controlled according to a time-varying suction profile. As used herein, the term “time-varying suction profile” refers to a suction control signal or function that varies as a function of time to regulate the magnitude, duration, and pattern of negative pressure generated by the suction mechanism.

The time-varying suction profile may comprise periodic, quasi-periodic, or non-periodic functions, including pulse-based, intermittent, hybrid, asymmetric, or nonlinear waveforms. In certain embodiments, the time-varying suction profile is represented as a function S(t), where suction strength varies over time according to one or more parameters including amplitude, frequency, duty cycle, phase, or asymmetry.

In certain embodiments, the time-varying suction profile is dynamically adjusted by the control circuitry based on sensor inputs, patient classification, and safety constraints.

### Pulse Suction:

In an embodiment, when the user selected pulse suction pattern, the control program of the embodiment will implement pulse suctions according to selected patient type. To let the suction strength oscillates periodically mimicking the effects of natural coughing reflex, the pulse suction strength is modeled by a sinusoidal function

$$S ( t ) = {S}_{max} \cdot {sin}^{2} ( \frac{2 π t}{T} )$$

Where:
S(t) is the suction strength at time t.
Sₘₐₓ is the maximum suction strength which depends on the user selected patient type and suction strength level for selected patient type and may vary for a specific design of an embodiment. When integrated with real-time sensor feedback, Sₘₐₓ can also be adjusted dynamically within the safe range during the operation.
T is the duration of one full cycle of pulse suction.

In other embodiment, the pulse suction can be further modeled with a time scaling factor that adjusts the frequency or period of suction pulses:

$$S ( t ) = {S}_{max} \cdot {e}^{- β t} \cdot {sin}^{2} ( 2 π t f ( t ) )$$

Where f(t) is a function that dynamically adjusts the frequency of pulses. For other embodiment, we can let the frequency to increase or decrease over time by setting f(t) as:

$$f ( t ) = α t + {f}_{0}$$

Here:
f₀ is the initial frequency of suction pulses.
α controls how quickly the pulse frequency increases or decreases over time
β is the decay factor, controlling how quickly the suction strength diminishes over time.

In other embodiment, if we want more abrupt transitions in suction strength, a triangular wave instead of a sinusoidal wave can be used for the suction strength:

$$S ( t ) = {S}_{max} \cdot {sin}^{2} ( 1 - \vert \frac{2 t}{T} - 1 \vert )$$

### Intermittent Suction:

In an embodiment, when the user selected intermittent suction pattern, the control program of the embodiment will implement intermittent suctions according to selected patient type and suction level. Suction is applied continuously for a preset duration, on-time, for example, 3-5 seconds. After this period, the suction briefly stops, for example, off-time, for 1-2 seconds. This on-off cycle repeats for several cycles until the obstruction is cleared or the user stops the device, or the control program stops the device based on sensor feedback.

In an embodiment, the suction strength during the on-time of intermittent suction may follow a trapezoidal pattern, ensuring smooth ramp-up and ramp-down transitions:

$$S ( t ) = \{ \begin{matrix}\frac{{S}_{max}}{{T}_{r}} \cdot t , & 0 \leq t \leq {T}_{r} \\ {S}_{max} , & {T}_{r} < t \leq {T}_{on} - {T}_{f} \\ {S}_{max} \cdot \frac{{T}_{on} - T}{{T}_{f}} & {T}_{on} - {T}_{f} < t \leq {T}_{on} \\ 0 , & t > {T}_{on}\end{matrix}$$

Where
S(t) is the suction strength at time t.
Sₘₐₓ is the maximum suction strength, which varies according to selected patient type, selected suction strength for selected patient type, and design of a specific embodiment. It can also be adjusted dynamically based on real-time sensor feedback.
Tᵣ is the ramp-up time (how long it takes for suction to reach maximum suction strength).
T_{f} is the ramp-down time (how long it takes for suction to decrease to zero).
Tₒₙ is the total duration of the on-phase (suction time)

In other embodiment, a simpler approach during the on-time can use a linear model that increases and decreases the suction strength linearly, without the steady-state phase as the trapezoidal model:

$$\{ \begin{matrix}{S}_{max} \cdot \frac{t}{{T}_{on}} , & 0 \leq t \leq {T}_{on} / 2 \\ {S}_{max} \cdot \frac{{T}_{on} - t}{{T}_{on} / 2} , & {T}_{on} / 2 < t \leq {T}_{on} \\ 0 , & t > {T}_{on}\end{matrix}$$

### Hybrid Suction:

In this mode, the function for suction strength will be a combination of strength function of pulse suction and intermittent suction. In this dual mode, the suction is applied in short, controlled bursts (pulses), with pauses between each set of pulses to create intermittent cycles of activity and rest. During the on-time, the pulse intensity, frequency will take the form of a pulse suction, and the on-off duration will take the form of intermittent suction. The following is one of such combinations:

$$\{ \begin{matrix}{S}_{max} \cdot {e}^{- β t} \cdot {sin}^{2} ( 2 π tf ( t ) ) & 0 \leq t \leq {T}_{on} \\ 0 , & t > {T}_{on}\end{matrix}$$

Where Sₘₐₓ, β, f(t), Tₒₙ are similarly defined as in pulse and intermittent suctions. Specific form of functions and related parameters will be determined by a specific embodiment.

### 9. Real-Time Adjustments of Suction Strength

The control program continuously monitors sensor feedback (32), (42) from pressure and motion sensors and adjusts suction strength. When obstruction is cleared, or abnormal pressure is detected, the control program stops operation accordingly.

In an embodiment, maximum strength Sₘₐₓ may be dynamically adjusted based on pressure and motion sensor feedback. For example, the Sₘₐₓ can be replaced by a function

$$SM ( t ) = {S}_{base} + {K}_{p} \cdot ( {P}_{target} - P ( t ) ) + {K}_{m} \cdot M ( t )$$

Where:
S(t) is the suction strength at time t,
S_{base} is the base suction strength (the minimum suction strength applied). The device starts at the base strength that is safe for selected patient type.
P_{target} is the target pressure (desired negative pressure in the airway) based on the patient type.
P(t) is the real-time pressure reading from the sensor at time t.
M(t) is a motion factor based on motion sensor data (0 if no motion, 1 if motion detected),
Kₚ and Kₘ are proportional control constants (tuning factors that determine how much the pressure and motion inputs affect the suction strength).

The difference between the target pressure P_{target} and the real-time measured pressure P(t) drives how much the suction strength should be adjusted. If the real-time pressure reading P(t) is lower than the desired value P_{target}, it indicates that the suction strength might not be strong enough to dislodge the obstruction. The control program increases S(t) (suction strength) proportionally to the difference between the actual and target pressure values. If P(t) is too high (indicating excessive suction), the algorithm decreases S(t) to avoid airway damage.

Above SM(t) can then be used as Sₘₐₓ with both pulse suction and intermittent suction. For example, the general strength function

$$S ( t ) = SM ( t ) \cdot {e}^{- β t} \cdot {sin}^{2} ( 2 π tf ( t ) )$$

For a pulse suction can be changed into

$$S ( t ) = ( {S}_{base} + {K}_{p} \cdot ( {P}_{target} - P ( t ) ) + {K}_{m} \cdot M ( t ) \cdot {e}^{- β t} \cdot {sin}^{2} ( 2 π tf ( t ) )$$

Similar function can be obtained for the intermittent suction mode.

In an embodiment, such functions will be used in (35) to adjust suction strength based on sensor feedback.

### 10. Sensor Monitoring and Workflow Logic

The device is equipped with integrated pressure and motion sensors within air chambers continuously monitor the air pressure and motions. In an embodiment, these sensors may be placed as (10) and (11) within the suction chamber (5) and before the filters (8), (19). This real-time feedback allows the device to dynamically adjust the suction operation to ensure proper positioning of the device and safety. An embodiment can implement, not limited to, the following workflows:

In an embodiment, when the device is powered on (30), (40), the control program starts suction operation (31), (41) according to selected patient type and suction pattern. The control program continues monitoring sensor feedback (32), (42). The control program automatically adjusts suction strength and patters in real-time on sensor feedback, ensuring optimal operation and patient safety. The following steps in an embodiment take into account of feedback from both pressure sensors and motion sensors:
Abnormal Air Pressure (33): If the sensors detect low air pressure in the air chambers (3), (5), this indicates that the device is either not correctly positioned on the patient's face or the seal is incomplete. If air pressure in the air chambers (3), (5) exceeds predefined threshold, it indicates risk of excessive force that could cause airway injury. In either case, the control program will stop suction operation (37) to ensure patient safety.
Air pressure within Safe Range (34): When the air pressure in the air chambers (3) and (5) is within the safe range, the control program may adjust suction strength (35) based on sensor feedback and continue suction operation (31).
Sudden Drop in Air Pressure (36): A sudden drop in air pressure in the air chambers (3) and (5) may not exceed the safe range, but it may be an indication of obstruction has been cleared. The control program will immediately cease suction (37) to minimize the risk of injury or discomfort.
No Object Motion Detected (43): If no motion is detected in the air chamber (3) and (5), the system continues suction (41).
Object Motion Detected (44): If motion is detected in the air chamber (3) and (5), the system ceases suction (45) and power off (46) automatically to avoid unnecessary force, minimizing the risk of injury or discomfort.

### 11. Design and Calibration for Safety and Effectiveness:

As an airway suction device, it is a challenge to be effective and safe. The device's components, including the facemask (1), air chamber (3), (5), (21), and sensors (10), (11), need to be carefully calibrated to accommodate various patient anatomies and usage scenarios. Suction power and the algorithms will be fine-tuned to be strong enough to effectively remove obstructions while maintaining the safety ranges to avoid injury. Calibration settings for suction patterns, timing, duration, and suction levels of an embodiment should be based on experimental data and medical guidelines to ensure safety and optimal performance.

### 12. Portability and Storage

The device is designed with portability in mind, ensuring it can fit easily into small bags or carrying cases. The modular components, including the facemask (1) and air chambers (3), (5), (21), can be quickly disassembled for easy storage. The lightweight design, combined with detachable parts, ensures the device is easy to carry and ready for use, suitable for parents, caregivers, restaurant owners, or medical professionals in various environments.

In addition to the embodiments described above, further embodiments are provided as follows.

### Generalized Negative Pressure Generation

In certain embodiments, the suction mechanism comprises a diaphragm pump configured to generate negative pressure through reciprocating membrane motion.

In certain embodiments, the suction mechanism may alternatively comprise a piston pump, rotary pump, Venturi system, or combinations thereof.

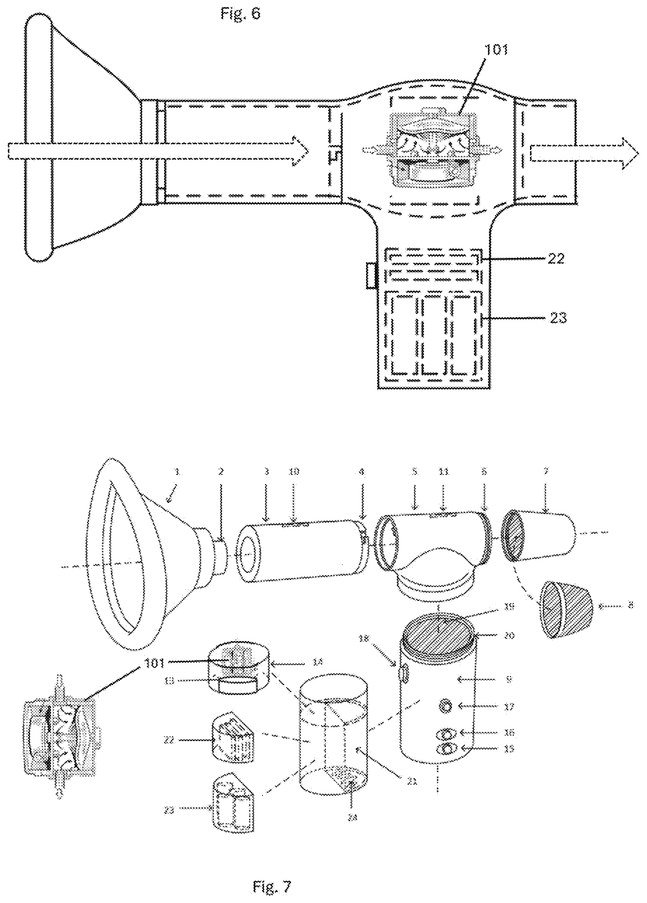

Referring to FIG. 6, an embodiment utilizing a diaphragm pump 101 is illustrated,

Referring to FIG. 7, an embodiment utilizing a diaphragm pump 101 is illustrated.

### Alternative Airflow Pathway Configurations

Referring to FIG. 6, in certain embodiments, airflow travels in a substantially linear path from the facemask to the distal end of the device.

In certain embodiments, airflow does not require a directional change exceeding 45 degrees.

In certain embodiments, the airflow pathway excludes debris filters.

### Waveform Modulation

In certain embodiments, suction strength is defined as:

$$S ( t ) = S\_max \cdot f ( t ; θ )$$

Where f(t; θ) is a nonlinear periodic function.

In certain embodiments, asymmetric waveforms are used with unequal rise and decay times.

### AI-Driven Patient Profiling

In certain embodiments, the system performs an initial micro-pulse suction to determine airway resistance.

Airway resistance is defined as:

$$R = Δ P / Q$$

The system classifies patient type and constrains suction within safe clinical ranges.

Referring to FIG. 8, an AI-driven patient profiling process is illustrated for determining patient classification and corresponding safety limits for suction operation. In certain embodiments, the process begins with powering on the device, as indicated at step 50, followed by applying an initial micro-pulse suction, as indicated at step 51. The initial micro-pulse suction is configured to generate controlled and limited negative pressure sufficient to characterize airway response without exceeding safety thresholds.

At step 52, the system measures airway characteristics, including pressure response and airflow, and determines an airway resistance parameter. In one example, airway resistance may be computed as:

$$R = Δ P / Q$$

where ΔP represents a pressure differential and Q represents airflow. Additional parameters, including compliance, transient pressure response, or airflow dynamics, may also be measured.

At step 53, the system computes a patient classification based on the measured airway characteristics. In certain embodiments, classification is performed using embedded software implementing rule-based logic, statistical models, or machine learning algorithms.

At decision step 54, the system classifies the patient into one of multiple categories, including infant (55), child (56), or adult (57). The classification may be determined based on thresholds, probabilistic outputs, or learned models derived from sensor data.

Following classification, the system sets patient-specific safety limits, as indicated at step 58. The safety limits may include maximum allowable suction pressure, waveform parameters, and control constraints corresponding to the classified patient category.

In certain embodiments, the patient classification and safety limits are used by the control circuitry to regulate subsequent suction operation, including waveform selection, amplitude constraints, and termination conditions. In alternative embodiments, the classification process may be repeated or updated dynamically based on additional sensor inputs during operation.

### Sensor Fusion

In certain embodiments, the system incorporates multiple sensing modalities, including pressure sensors, motion sensors, acoustic sensors, and optical sensors, to monitor suction conditions and detect airway clearance events.

In certain embodiments, a combined decision metric is computed based on outputs from one or more of the sensors to determine whether an obstruction has been dislodged or whether suction should be adjusted or terminated.

In one example, the combined decision metric D is defined as:

$$D = {w}_{1} P ( t ) + {w}_{2} M ( t ) + {w}_{3} A ( t ) + {w}_{4} O ( t )$$

where:
P(t) represents a pressure-based signal;
M(t) represents a motion-based signal;
A(t) represents an acoustic signal;
O(t) represents an optical detection signal;
w₁, w₂, w₃, w₄ are weighting factors.

In certain embodiments, the combined decision metric may alternatively comprise nonlinear functions, weighted thresholds, logical rules, or machine learning-based models that combine one or more sensor signals.

In certain embodiments, suction is modified or terminated when the combined decision metric exceeds a predefined threshold.

In alternative embodiments, the combined decision metric may be computed using rule-based logic, threshold comparisons, machine learning models, or other data fusion techniques, and need not be limited to a linear combination of sensor signals.

In certain embodiments, the device includes control circuitry and embedded software configured to regulate operation of the suction mechanism based on user inputs and real-time sensor feedback.

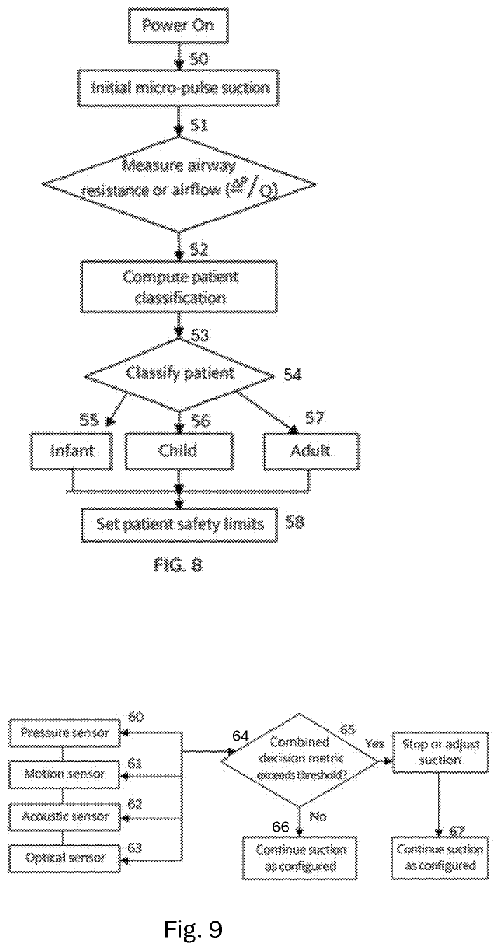

Referring to FIG. 9, a sensor fusion-based decision process is illustrated for controlling suction operation. In certain embodiments, the system receives input signals from a plurality of sensors, including a pressure sensor 60, a motion sensor 61, an acoustic sensor 62, and an optical sensor 63. These sensor signals are provided to a decision module 64, which computes a combined decision metric based on one or more of the sensor inputs.

The decision module evaluates whether the combined decision metric exceeds a predefined threshold at decision step 65. The combined decision metric may be derived from a weighted combination of sensor signals, threshold comparisons, or other data fusion techniques, as described herein.

If the combined decision metric exceeds the threshold, the system determines that an airway clearance event or abnormal condition has occurred, and the control circuitry generates a control signal to stop or adjust suction, as indicated at step 65. In certain embodiments, suction may be reduced, paused, or terminated depending on the detected condition.

If the combined decision metric does not exceed the threshold, the system continues suction operation as configured, as indicated at step 66. In certain embodiments, even after suction is stopped or adjusted, the system may resume or continue suction in a controlled manner, as indicated at step 67, based on updated sensor inputs or control logic.

In alternative embodiments, the decision module 64 may implement rule-based logic, proportional control, or machine learning models to determine whether to modify or terminate suction, and the specific form of the combined decision metric is not limited to any particular mathematical expression.

### Improved Control Logic & Embedded Software

In certain embodiments, the device includes control circuitry and embedded software configured to regulate operation of the suction mechanism based on user inputs and real-time sensor feedback.

The control circuitry is operatively coupled to the suction mechanism, sensors, and user interface elements, and is configured to generate control signals that determine suction strength, waveform characteristics, and operation timing.

### Closed-Loop Control Framework

In certain embodiments, the system operates as a closed-loop control system in which suction output is dynamically adjusted based on measured system conditions.

In one example, suction strength S(t) is determined as:

$$S ( t ) = {S}_{base} + {k}_{1} ( {P}_{target} - P ( t ) ) + {k}_{2} M ( t ) + {k}_{3} A ( t ) + {k}_{4} O ( t )$$

where:
S_{base} is a baseline suction level;
P(t) is a measured pressure;
P_{target} is a target pressure;
M(t), A(t), and O(t) represent motion, acoustic, and optical signals;
k₁, k₂, k₃, k₄ are control gains.

### Safety-Constrained Control

In certain embodiments, the control system constrains suction output according to patient-specific safety limits:

$$S ( t ) \leq {S}_{safe}$$

where S_{safe} corresponds to predefined clinical ranges associated with patient classification.

### Waveform-Integrated Control

In certain embodiments, the control system generates a time-varying suction profile:

$$S ( t ) = {S}_{max} \cdot f ( t ; θ )$$

where f(t; θ) is a nonlinear periodic function.

The control circuitry may dynamically adjust parameters θ, including frequency, amplitude, duty cycle, and asymmetry, based on sensor feedback.

### AI-Assisted Adaptive Control

In certain embodiments, the control system includes a classification module configured to determine patient type based on measured airway response.

The classification may be represented as:

$$C = arg {max}_{i} P ( {class}_{i} \vert sensor\_data )$$

The control system uses the classification result to:
set safe suction limits
select waveform parameters
adjust control gains

### Event Detection and Termination Logic

In certain embodiments, the control system determines whether to modify or terminate suction based on a decision metric derived from sensor signals.

Suction may be terminated when:
obstruction clearance is detected
abnormal pressure conditions occur
leakage or improper seal is detected

### ALTERNATIVE IMPLEMENTATIONS

In alternative embodiments, the control system may be implemented using:
proportional-integral-derivative (PID) control
rule-based logic
state machines
machine learning models
or combinations thereof.

The specific control strategy may vary without departing from the scope of the invention,

1. A negative pressure airway clearance system comprising:
(a) a facemask configured to form an airtight seal;
(b) an airflow pathway connecting the facemask to a suction mechanism;
(c) a suction mechanism configured to generate negative pressure, wherein the suction mechanism comprises at least one of a propeller system, a diaphragm pump, a piston pump, a Venturi system, or combinations thereof; and
(d) control circuitry configured to modulate suction according to a time-varying suction profile;
wherein the time-varying suction profile comprises a nonlinear periodic function; and
wherein the control circuitry is further configured to dynamically adjust the time-varying suction profile based on a combined decision metric derived from a plurality of sensor inputs.

2. The system of claim 1, wherein the airflow pathway comprises a substantially linear flow path without a 90-degree directional change.

3. The system of claim 1, wherein the suction mechanism comprises a diaphragm pump configured to generate negative pressure through reciprocating membrane motion.

4. The system of claim 3, wherein the diaphragm pump is driven at a variable frequency to produce the time-varying suction profile.

5. The system of claim 1, wherein the time-varying suction profile comprises an asymmetric waveform having a rise time different from a decay time.

6. The system of claim 1, wherein the time-varying suction profile comprises a variable-duty-cycle waveform.

7. The system of claim 1, wherein a duration of peak suction is less than 40% of a total cycle.

8. The system of claim 1, wherein the time-varying suction profile comprises a time-varying frequency.

9. The system of claim 1, wherein the time-varying suction profile further comprises a stochastic perturbation component.

10. The system of claim 1, further comprising at least one sensor selected from pressure sensors, motion sensors, acoustic sensors, and optical sensors.

11. The system of claim 10, wherein the combined decision metric is derived from at least two different types of sensor signals.

12. The system of claim 10, wherein suction termination or adjustment is based on the combined decision metric.

13. The system of claim 10, further comprising an acoustic sensor configured to detect airway clearance events.

14. The system of claim 10, further comprising an optical sensor configured to detect passage of debris.

15. The system of claim 1, further comprising embedded software configured to automatically classify patient type based on airway response and constrain suction within predefined clinical ranges.

16. The system of claim 15, wherein classification is determined using a machine learning model.

17. The system of claim 15, wherein the time-varying suction profile is selected or modified based on the patient classification.

18. The system of claim 1, wherein suction strength is adjusted based on a difference between a target pressure and a measured pressure in a closed-loop control system.

19. A method for clearing an airway obstruction, comprising:
(a) forming an airtight seal using a facemask;
(b) generating negative pressure using a suction mechanism;
(c) modulating suction using a time-varying suction profile;
(d) receiving sensor inputs from a plurality of sensors;
(e) computing a combined decision metric based on the sensor inputs;
(f) dynamically adjusting suction based on the combined decision metric; and
(g) terminating or modifying suction upon detection of a clearance or safety condition.

20. A non-transitory computer-readable medium storing instructions that, when executed by control circuitry of a negative pressure airway clearance system, cause the system to:
(a) generate a nonlinear time-varying suction profile;
(b) receive sensor inputs;
(c) compute a combined decision metric based on the sensor inputs;
(d) adjust suction based on the combined decision metric and a difference between a target pressure and a measured pressure;
(e) constrain suction within a predefined safety range; and
(f) control a suction mechanism accordingly.
